(19) United States
(12) Patent Application Publication
(10) Pub. No.: US 2005/0277946 A1
(43) Pub. Date: Dec. 15, 2005

(54) Access port for laparoscopic surgery

(75) Inventor: E. Skott Greenhalgh, Wyndmoor, PA (US)

Correspondence Address: SYNNESTVEDT & LECHNER, LLP, 2600 ARAMARK TOWER, 1101 MARKET STREET, PHILADELPHIA, PA, 191072950, US

(73) Assignee: Secant Medical, LLC, Perkasie, PA (US)

(21) Appl. No.: 11/152,434

(22) Filed: Jun. 14, 2005

Related U.S. Application Data
(60) Provisional application No. 60/579,614, filed on Jun. 15, 2004.

(51) Int. Cl. A61B 17/10

(52) U.S. Cl. 606/108

(57) ABSTRACT

An access port for use in laparoscopic surgery is disclosed. The port includes a duct having a one-way valve and a tubular seal. The one-way valve has opposed surfaces that co-apt in response to internal pressure within the duct. The tubular seal has an inner layer with a low friction coefficient surrounded by an outer elastic layer that biases the inner layer into sealing engagement with a surgical tool inserted through the duct. The one-way valve seals the duct in the absence of a tool extending through the duct. The low friction coefficient of the inner layer facilitates insertion and removal of the tool through the duct. The port has a distal end insertable into a pressurized cavity, and a proximal end that extends from the cavity and provides access thereto.

### FIELD OF THE INVENTION

The invention concerns an access port having a flexible resilient tube with an inner surface which substantially conforms to an outer surface of an item inserted through the port to provides a fluid-tight seal between the tube and the item.

### BACKGROUND OF THE INVENTION

Various medical procedures require that sealing access ports be provided for the introduction and removal of surgical tools, guide wires, catheters or other items into the cavity or vessel being operated upon. A sealing access port is necessary when the procedure is carried out in a region of higher pressure within the body which must be maintained at that pressure without allowing significant leakage. For example, in a laparoscopic procedure within the abdomen, carbon dioxide gas is pumped into the abdomen to form an expanded or enlarged cavity within which the procedure may be carried out. As various tools are inserted and removed through the access ports, it is advantageous that the ports seal substantially fluid-tight to maintain the gas pressure within the cavity and keep it inflated.

Similarly, a sealing access port, more properly called an “introducer”, is advantageous when catheters and guide wires are being inserted within a pressurized vessel, such as an artery of the vascular system, to prevent blood from leaking out as the items are introduced and removed.

Access ports currently provide a seal that prevents leakage when there is no tool inserted through the port. However, when a tool or other item is inserted through the port and manipulated, the seals as currently configured cannot maintain sufficient integrity to prevent significant leakage. It would be advantageous to provide an access port or an introducer that provides an adequate fluid-tight seal under all conditions of use, i.e., when a tool or other device is absent from the port, as well as when a tool or item extends through the port into the cavity or vessel which is the subject of the procedure.

### SUMMARY OF THE INVENTION

The invention concerns an access port useable to perform procedures within a pressurized environment, for example, within a body cavity during laparoscopic surgery or within a vascular vessel. The access port permits insertion of a tool into the pressurized environment while substantially maintaining pressure therein. The access port comprises an elongated duct. A plurality of flexible, resilient opposed surfaces are positioned within the duct. The opposed surfaces cooperate with one another when subjected to internal pressure within the duct to form a substantially fluid-tight one-way valve. The opposed surfaces are flexibly deformable to permit the tool to be inserted through the duct and into the pressurized environment.

A flexible, resilient tube is in fluid communication with the duct. The tube is radially inwardly biased so as to be engageable with the tool and form a substantially fluid-tight seal therearound when the tool is inserted through the duct and the tube. In one embodiment, particularly suited for use in laparoscopic surgery, the tube is positioned within the duct and the duct has a distal end insertable through an opening in the living tissue surrounding the pressurized body cavity. The duct has an outer surface that forms a seal with the living tissue. A proximal end of the duct extends outwardly from the cavity to receive the tool.

In another embodiment, suitable for procedures in vascular vessels, the tube is attached to the duct in end to end relationship.

Preferably, the tube comprises an inner layer having a low coefficient of friction. The inner layer interfaces with the tool and facilitates insertion of the tool therethrough. An outer elastic layer surrounds the inner layer. The outer elastic layer provides the radially inward biasing that enables the tube to form a substantially fluid-tight seal around the tool.

The tube may have one of a number of different profile shapes, for example, the tube may be substantially cylindrical in profile, have a substantially hourglass-shaped profile or a tapered profile.

### BRIEF DESCRIPTION OF THE DRAWINGS

FIGS. 1 and 2 are longitudinal sectional views of an access port according to the invention;

FIG. 3 is a longitudinal sectional view of a component of an access port, the component having a substantially cylindrical profile;

FIG. 4 is a longitudinal sectional view of a component of an access port, the component having a substantially hourglass shaped profile;

FIG. 5 is a longitudinal sectional view of a component of an access port, the component having a substantially tapered profile;

FIG. 6 is a longitudinal section view of a component of an access port according to the invention;

FIGS. 7-9 are side views of a component of an access port according to the invention illustrating, respectively, braided, knitted, and woven embodiments thereof;

FIG. 10 is a longitudinal sectional view of an embodiment of an access port according to the invention; and

FIGS. 11-14 illustrate the use of the access port shown in FIG. 10 in a procedure within a vascular vessel.

### DETAILED DESCRIPTION OF THE EMBODIMENTS

FIG. 1 shows a sealing access port 10 according to the invention useable in laparoscopic surgical procedures. Access port 10 comprises an elongated duct 12 surrounding and defining a bore 14. Duct 12 preferably has a substantially rigid sidewall 16 that allows the access port 10 to be inserted into a body cavity 20 through an opening in a muscular tissue wall 22 and maintain the patency of the access port 10 against the muscle pressure during the laparoscopic procedure. The outer surface 18 of the sidewall 16 forms a seal with the living tissue 22 to maintain a higher pressure within the body cavity 20 as described below.

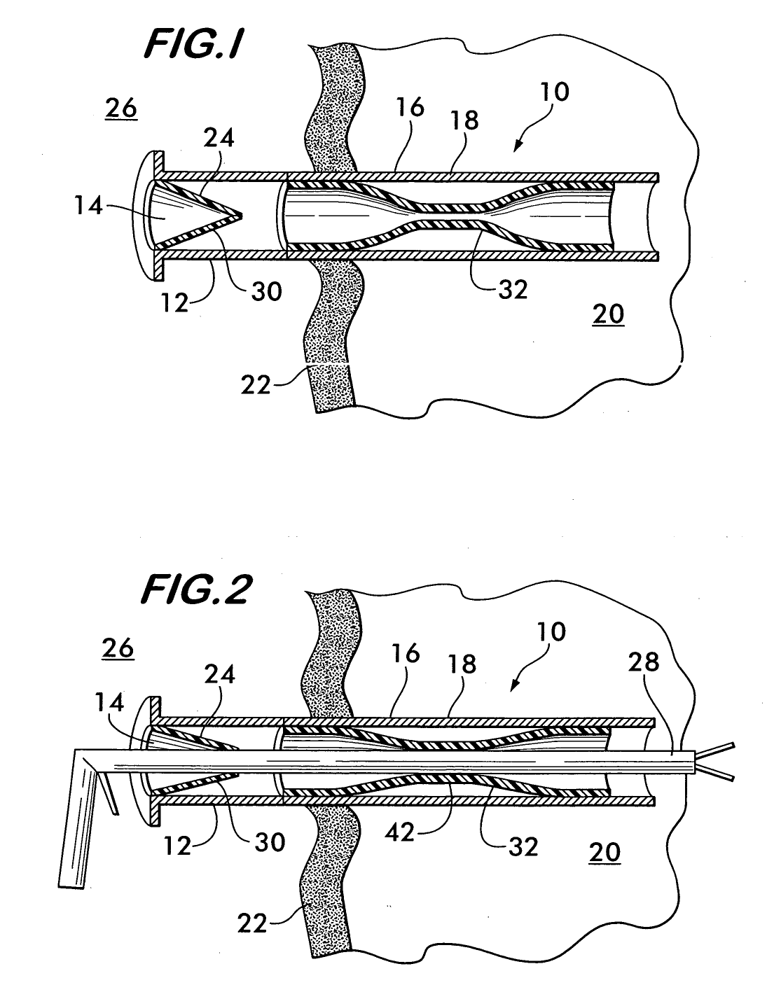

A one-way valve 24 is positioned within the duct 12, preferably at a proximal end that extends outwardly from the cavity 20. One-way valve 24 is designed to close substantially fluid-tight in response to a pressure differential between the body cavity 20 and the ambient 26. The differential pressure results when the cavity-is pressurized with gas to provide an enlarged space to perform the surgical procedure. As shown in FIG. 2, the one-way valve 24 also allows surgical tools 28 to extend through the duct 12 and into the body cavity 20. Thus, it is advantageous that the one-way valve 24 comprise resilient, flexible opposed surfaces 30, preferably in the form of a duckbill. As shown in FIG. 1, due to their duckbill shape and flexibility, the opposed surfaces 30 cooperate with one another, for example by co-apting, when subjected to the increased pressure within the body cavity 10 and provide a substantially fluid-tight seal preventing leakage to the ambient 26 through the duct 12. However, the opposed surfaces 30, being flexible, deform readily to allow the tool 28 to pass through the duct 12 and into the body cavity 20. Because they are resilient, the opposed surfaces 30 co-apt again upon removal of the tool 28 to effect the seal.

When tool 28 extends through one-way valve 24, it is not always possible for the valve to maintain a fluid-tight seal against the tool. This is especially true when the tool is manipulated to perform the procedure, the manipulations, usually consisting of angular displacements of the tool within the access port 10, cause the opposed surfaces 30 to separate and allow leakage from the pressurized body cavity 20 to the ambient 26. To prevent such leakage when tool 28 extends though the access port 10, a tubular seal 32 is installed within bore 14, preferably in a distal portion of the duct 12.

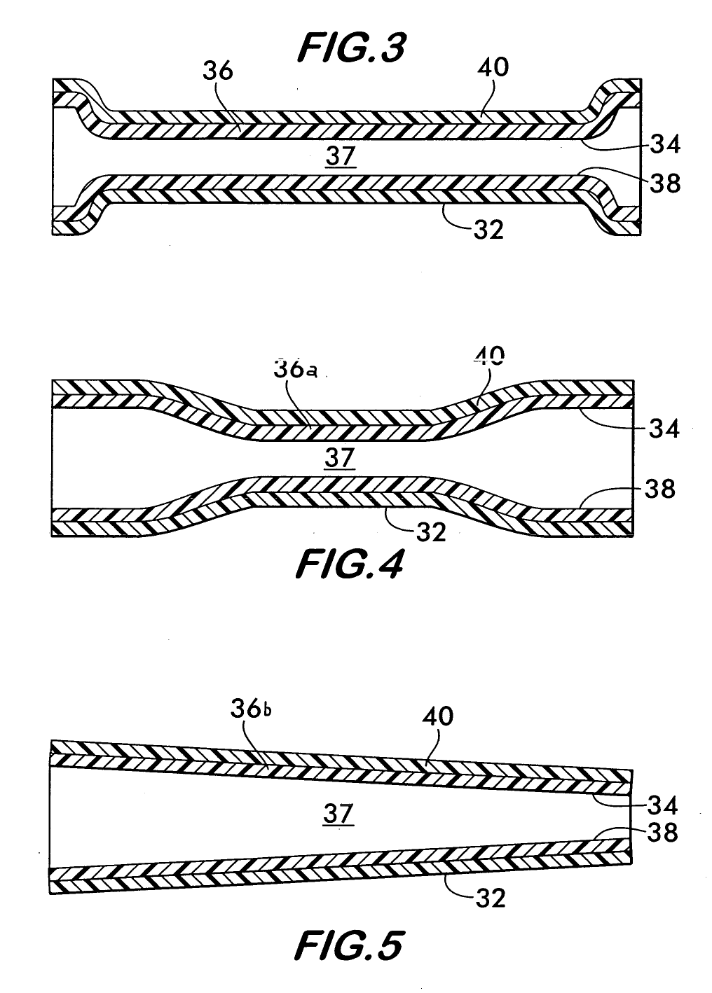

Tubular seal 32 is shown in detail in FIG. 3 and preferably comprises two layers. An inner layer 34, in this example having the shape of an elongated cylinder 36, surrounds and defines a central space 37 for receiving the tool 28. Inner layer 34 has a pliant, lubricious inner surface 38 that interfaces with the tool 28 to form a substantially fluid-tight seal preventing unacceptable leakage. The lubricious inner surface 38 has a low coefficient of friction that allows the tool 28 to pass easily through the tubular seal 32 with little friction. An outer elastic layer 40 surrounds the inner layer 34. Elastic layer 40 provides radial biasing that forces the inner surface 38 of inner layer 34 into sealing engagement with the tool 28. Together, the two layers 34 and 40 are radially expandable and contractible to accommodate tools 28 of various diameters and shapes. As readily observable in FIG. 2, the tubular seal 32 preferably interfaces with the tool 28 over a predetermined engagement length 42. Thus, its effectiveness as a seal is not adversely affected by motion of the tool 28 during the surgical procedure, the tubular seal 32 being flexible and following the motions of the tool while maintaining sealing contact between its inner surface 38 and the tool 28 over at least a portion of its engagement length 42. Although the tube is flexible in bending, radially compliant and resilient, it is advantageous that it also resist lengthwise stretching, i.e., it is preferred that the tube 32 be axially stiff.

Preferably, the inner layer 34 of tubular seal 32 is formed from expanded polytetrafluoroethylene (PTFE) and the outer elastic layer comprises an elastic membrane formed from elastic material such as rubber, polyurethane or silicone. Expanded PTFE is preferred because it provides the pliant, lubricious inner surface 38 having a low coefficient of friction as well as the desired mechanical properties of axial stiffness and radial compliance. Other polymers such as nylon, polyethylene, polypropylene and polyester are also feasible for inner layer 34.

The tubular seal 32 is formed by expanding a PTFE tube plastically beyond its yield point so that it takes a permanent set at a predetermined enlarged diameter, forming the inner layer 34. The expanded PTFE tube is then worked to compress it radially back down close to its original (smaller) diameter. Because of its plastic expansion, the expanded PTFE tube has lost its resilient, elastic qualities and will readily expand and contract radially between the smaller and larger diameters, but it will not of itself return to either diameter. The outer elastic layer 40 is positioned surrounding the inner layer 34 and resiliently biases the inner layer radially inwardly toward its smaller diameter. This combination of elastic and low friction layers allows the tubular seal 32 to provide a lubricious inner surface 38 that expands and contracts radially to accommodate the tool 28 while maintaining the inner surface in sealing contact with the tool. It is possible to tailor the radial biasing force to a predetermined value by choice of material with different elastic properties (i.e., types of urethane, silicone or other materials) as well as by adjusting the durometer and thickness of the elastic biasing layer 40 on the inner layer 34.

As shown in FIGS. 3-5, the tubular seal 32 may have a wide variety of shapes ranging from the substantially cylindrical shape 36 (FIG. 3) to the hour glass shape 36a (FIG. 4) or the tapered shape 36b (FIG. 5) as well as others. These shapes are formed by first expanding a PTFE tube to make expanded PTFE and then inserting one or more mandrels into it. The expanded PTFE tube is then compressed radially so that it takes on the shape of the mandrel, and the outer elastic biasing layer is applied to the outer surface of the tube. Upon curing, the elastic biasing layer holds the tube in the preferred shape dictated by the mandrels, which are then removed. Application of the outer elastic biasing layer may be effected by dipping, spraying or injection molding as well as other procedures.

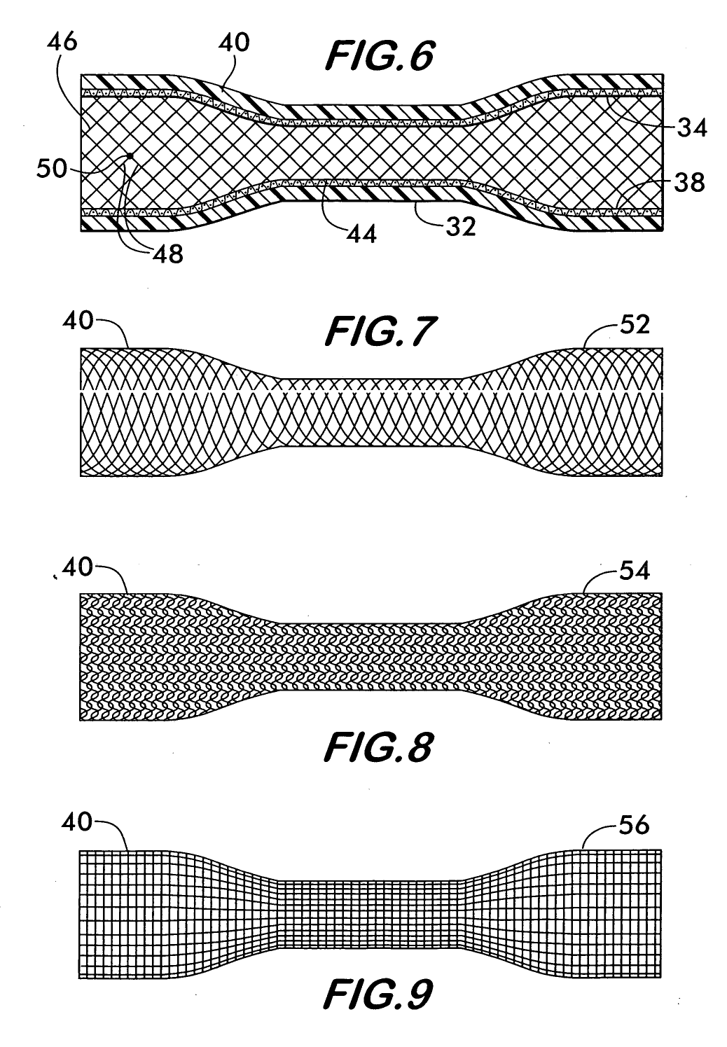

FIG. 6 illustrates an alternate embodiment of the tubular seal 32 wherein the inner layer 34 comprises a tube 44 formed from a lattice 46 of elongated members 48 joined to one another at intersection points 50. When made from PTFE or other lubricious polymers, lattice 46 provides an inner lubricious surface 38 as well as a structure that is radially expandable and contractible (but still longitudinally stiff) to accommodate tools of various sizes. Such a tube may be formed, for example, by starting with a solid tube and forming slots, slits or removing the interstitial material between the lattice members. With this procedure, the full panoply of stent manufacturing techniques may be utilized, as the tube resembles a stent in its physical behavior of radial compliance and axial stiffness. The slots, slits and removed material may have predetermined shapes as is known in stent manufacturing to imbue the tube with specific physical characteristics such as axial stiffness, radial compliance, bending stiffness and the like, all controllable by the shape and spacing of the openings in the tube. Cutting may be effected by laser, cutting blades, or a particular pattern of lattice may be molded. It is also possible to braid elongated members together and fuse them to one another at the intersection points.

The elastic outer biasing layer 40 surrounds the lattice 46 and forces it into engagement with the tool as described above for the previous examples. Biasing layer 40 in this case however, also performs the sealing function since the lattice 46 is a substantially open network in order to achieve the desired radial expansion and contraction characteristics. Once again, elastic flexible coatings or membranes such as rubber, polyurethane and silicone are preferred for the biasing layer 40.

Outer biasing layer 40 may also have alternate embodiments as illustrated in FIGS. 7-9. FIG. 7 shows a braided sleeve 52 that may be positioned surrounding an inner layer to form a multi-layer tubular seal. Similarly, FIG. 8 shows a knitted sleeve 54 and FIG. 9 shows a woven sleeve 56, either of which may surround an inner layer having a low friction surface to form a biased tubular seal. Preferably, the sleeves 52, 54 and 56 are formed from interlaced elastic filamentary members that allow them to expand and contract radially while biasing the inner layer sealingly against a tool. Elastic fibers such as lycra, hytrel and other synthetic polymers are preferred.

FIG. 10 shows an access port 58, also known as an introducer, which is preferred for use in vascular procedures. Introducer 58 is similar to access port 10 in that it has a duct 12 surrounding a bore 14 and a one-way valve 24 positioned within the bore 14 of the duct. A tubular seal 32 is attached to the duct in fluid communication with the bore 14. Preferably the tubular seal 32 has an inner layer 34 in the form of a tube having a lubricious inner surface 38, and an outer elastic biasing layer 40 surrounding the inner layer. Missing is the distal portion of duct 12 which is not used for vascular procedures.

FIGS. 11-14 show the introducer 58 in operation. A needle 60 is positioned within the bore 14 of the duct 12, the needle 60 extending through the central space 37 of the tubular seal 32. The elastic layer 40 biases the inner layer 34 against the needle 60 and forms a fluid-tight seal. The needle 60 with the introducer 58 is inserted through tissue 62 and into a blood vessel 64. The seal between the inner layer 34 and the needle 60 substantially prevents blood from escaping through the introducer to the ambient 26.

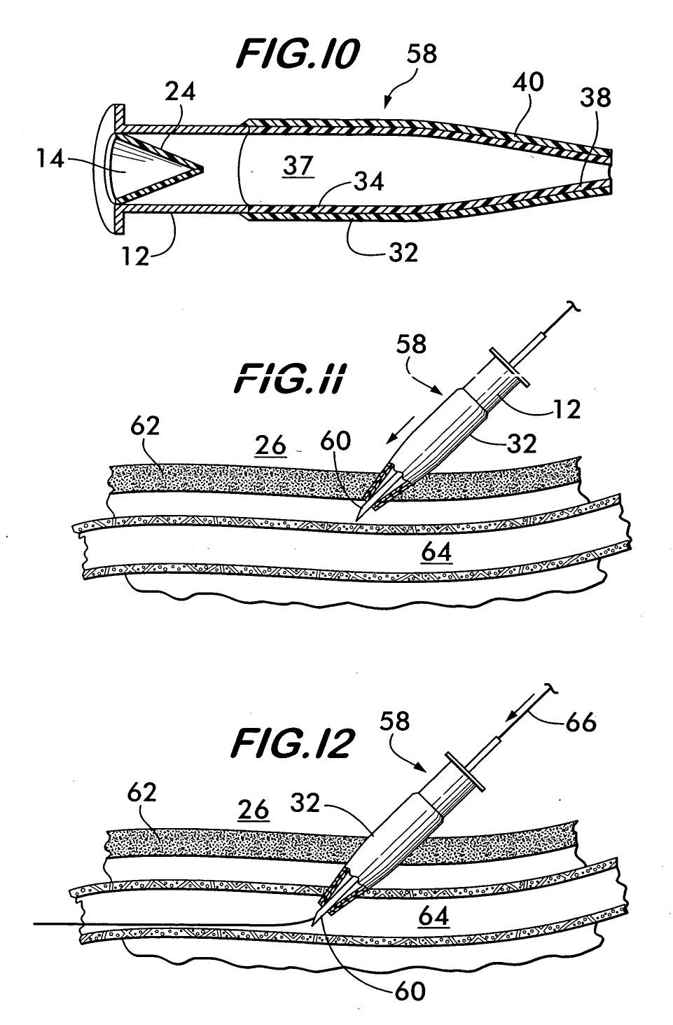

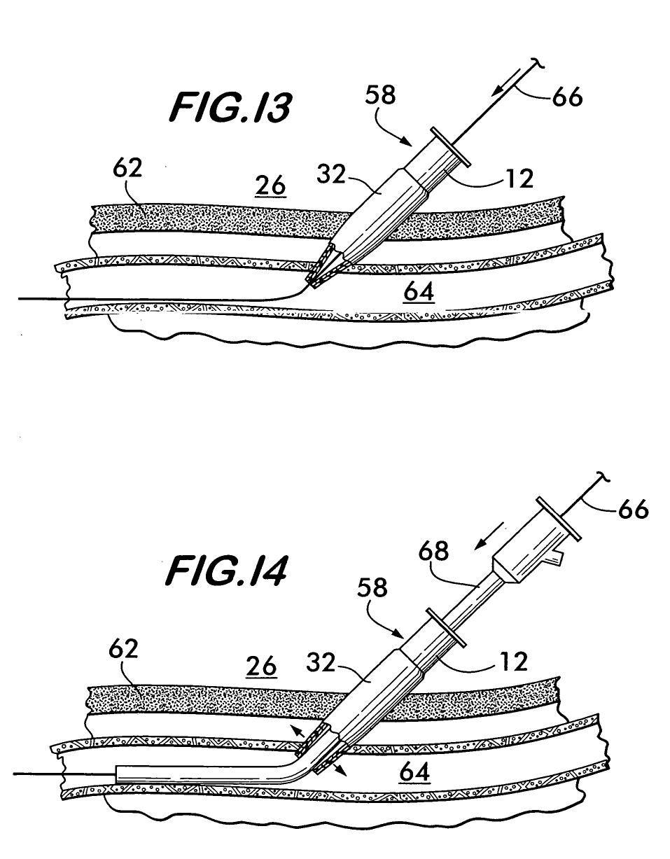

As shown in FIG. 12, a guide wire 66 may then be inserted into the vessel 64 through the needle 60. Next, as shown in FIG. 13 the needle is removed. Blood from the vessel 64 is prevented from leaking from the introducer 58 to the ambient by the one-way valve 24 positioned within the proximal portion of duct 12. As shown in FIG. 14 the introducer is used to insert items, such as catheter 68 into the vessel. The tubular seal 32 expands radially and sealingly engages catheter 68 and prevents any blood flow through the introducer 58 to the ambient 26.

Access ports according to the invention permit procedures to be performed within a pressurized environment while substantially maintaining the pressure through the use of multiple seals which cooperate to allow tools to be inserted and removed to and from the environment without significant leakage through the access port.

1. An access port useable for laparoscopic surgery within a pressurized body cavity surrounded by living tissue, said access port permitting insertion of a surgical tool into said cavity while maintaining pressure therein, said access port comprising:
an elongated duct having a distal end insertable through an opening in said tissue into said cavity, and a proximal end extending outwardly therefrom, said duct having an outer surface forming a seal with said living tissue;
a plurality of flexible, resilient opposed surfaces positioned within said duct, said opposed surfaces cooperating with one another when subjected to internal pressure within said duct to form a substantially fluid-tight one-way valve, said opposed surfaces being flexibly deformable to permit said tool to be inserted through said duct and into said cavity; and
a flexible, resilient tube positioned within said duct, said tube being radially inwardly biased so as to be engageable with said tool and form a substantially fluid-tight seal therearound when said tool is inserted through said duct.

2. An access port according to claim 1, wherein said tube has a predetermined engagement length over which it engages said tool.

3. An access port according to claim 1, wherein said opposed surfaces are positioned at said proximal end of said duct.

4. An access port according to claim 1, wherein said tube comprises an inner layer having a low coefficient of friction, said inner layer interfacing with said tool and facilitating insertion of said tool therethrough, an outer elastic layer surrounding said inner layer, said outer elastic layer providing said radially inward biasing.

5. An access port according to claim 4, wherein said inner layer comprises a material selected from the group consisting of expanded polytetrafluoroethylene, polypropylene, polyester and nylon.

6. An access port according to claim 4, wherein said outer elastic layer comprises an elastic membrane selected from the group consisting of rubber, polyurethane and silicone.

7. An access port according to claim 4, wherein said outer layer comprises a plurality of interlaced elastic filamentary members.

8. An access port according to claim 7, wherein said filamentary members are interlaced by a technique selected from the group consisting of braiding, weaving and knitting.

9. An access port according to claim 4, wherein said inner layer comprises a lattice of interconnected elongated members, and said outer elastic layer comprises an elastic membrane surrounding said inner layer.

10. An access port according to claim 1, wherein said tube has a substantially cylindrical profile.

11. An access port according to claim 1, wherein said tube has a substantially hourglass-shaped profile.

12. An access port according to claim 1, wherein said tube has a tapered profile.

13. An access port useable for procedures within a vascular vessel, said access port permitting insertion of a tool into said vessel while maintaining pressure therein, said access port comprising:
an elongated duct;
a plurality of flexible, resilient opposed surfaces positioned within said duct, said opposed surfaces cooperating with one another when subjected to internal pressure within said duct to form a substantially fluid-tight one-way valve, said opposed surfaces being flexibly deformable to permit said tool to be inserted through said duct and into said vessel; and
a flexible, resilient tube extending from an end of said duct, said tube being radially inwardly biased so as to be engageable with said tool and form a substantially fluid-tight seal therearound when said tool is inserted through said duct and into said vessel.

14. An access port according to claim 13, wherein said tube has a predetermined engagement length over which it engages said tool.

15. An access port according to claim 13, wherein said tube comprises an inner layer having a low coefficient of friction, said inner layer interfacing with said tool and facilitating insertion of said tool therethrough, an outer elastic layer surrounding said inner layer, said outer elastic layer providing said radially inward biasing.

16. An access port according to claim 15, wherein said inner layer comprises a material selected from the group consisting of expanded polytetrafluoroethylene, polypropylene, polyester and nylon.

17. An access port according to claim 15, wherein said outer elastic layer comprises an elastic membrane selected from the group consisting of rubber, polyurethane, and silicone.

18. An access port according to claim 15, wherein said outer elastic layer comprises a plurality of interlaced elastic filamentary members.

19. An access port according to claim 18, wherein said filamentary members are interlaced by a technique selected from the group consisting of braiding, weaving and knitting.

20. An access port according to claim 15, wherein said inner layer comprises a lattice of interconnected elongated members.

21. An access port according to claim 20, wherein said outer elastic layer comprises an elastic membrane surrounding said inner layer.

22. An access port according to claim 13, wherein said tube has a substantially cylindrical profile.

23. An access port according to claim 13, wherein said tube has a tapered profile.

24. An access port useable to perform procedures within a pressurized environment, said access port permitting insertion of a tool into said environment while maintaining pressure therein, said access port comprising:
an elongated duct;
a plurality of flexible, resilient opposed surfaces positioned within said duct, said opposed surfaces cooperating with one another when subjected to internal pressure within said duct to form a substantially fluid-tight one-way valve, said opposed surfaces being flexibly deformable to permit said tool to be inserted through said duct and into said environment; and
a flexible, resilient tube in fluid communication with said duct, said tube being radially inwardly biased so as to be engageable with said tool and form a substantially fluid-tight seal therearound when said tool is inserted through said duct and said tube.

25. An access port according to claim 24, wherein said tube is positioned within said duct.

26. An access port according to claim 25, wherein said tube has a predetermined engagement length over which it engages said tool.

27. An access port according to claim 24, wherein said tube is attached to said duct in end-to-end relationship.

28. An access port according to claim 24, wherein said tube comprises an inner layer having a low coefficient of friction, said inner layer interfacing with said tool and facilitating insertion of said tool therethrough, an outer elastic layer surrounding said inner layer, said outer elastic layer providing said radially inward biasing.
